SEQUENCE LISTING (16 sequences)

SEQ ID NO: 1 - DNA, 15 bp, Artificial Sequence
  Primer
accagcagttggagg

SEQ ID NO: 2 - DNA, 15 bp, Artificial Sequence
  Primer
gtttagtagcaagcc

SEQ ID NO: 3 - DNA, 15 bp, Artificial Sequence
  Primer
agaggcgaccattgg

SEQ ID NO: 4 - DNA, 15 bp, Artificial Sequence
  Primer
cccatagatggtcgg

SEQ ID NO: 5 - DNA, 15 bp, Artificial Sequence
  Primer
attcacccacagagg

SEQ ID NO: 6 - DNA, 15 bp, Artificial Sequence
  Primer
caacatctatactgg

SEQ ID NO: 7 - DNA, 15 bp, Artificial Sequence
  Primer
caatgacatgtatgg

SEQ ID NO: 8 - DNA, 15 bp, Artificial Sequence
  Primer
taaggctctctagag

SEQ ID NO: 9 - DNA, 15 bp, Artificial Sequence
  Primer
cttaccaatagcctg

SEQ ID NO: 10 - DNA, 15 bp, Artificial Sequence
  Primer
gtgtctagaacagcc

SEQ ID NO: 11 - DNA, 14 bp, Artificial Sequence
  Primer
agtagatttctggc

SEQ ID NO: 12 - DNA, 15 bp, Artificial Sequence
  Primer
cacatagcactaagc

SEQ ID NO: 13 - DNA, 2578 bp, Homo sapiens
tctacacttaaaatgccaccagcagttggaggtccagttggatacacccccccagatggaggctggggctgggcagtggtaattggagctttcatttccatcggcttctcttatgcatttcccaaatcaattactgtcttcttcaaagagattgaaggtatattccatgccaccaccagcgaagtgtcatggatatcctccataatgttggctgtcatgtatggtggaggtcctatcagcagtatcctggtgaataaatatggaagtcgtatagtcatgattgttggtggctgcttgtcaggctgtggcttgattgcagcttctttctgtaacaccgtacagcaactatacgtctgtattggagtcattggaggtcttgggcttgccttcaacttgaatccagctctgaccatgattggcaagtatttctacaagaggcgaccattggccaacggactggccatggcaggcagccctgtgttcctctgtactctggcccccctcaatcaggttttcttcggtatctttggatggagaggaagctttctaattcttgggggcttgctactaaactgctgtgttgctggagccctcatgcgaccaatcgggcccaagccaaccaaggcagggaaagataagtctaaagcatcccttgagaaagctggaaaatctggtgtgaaaaaagatctgcatgatgcaaatacagatcttattggaagacaccctaaacaagagaaacgatcagtcttccaaacaattaatcagttcctggacttaaccctattcacccacagaggctttttgctatacctctctggaaatgtgatcatgttttttggactctttgcacctttggtgtttcttagtagttatgggaagagtcagcattattctagtgagaagtctgccttccttctttccattctggcttttgttgacatggtagcccgaccatctatgggacttgtagccaacacaaagccaataagacctcgaattcagtatttctttgcggcttccgttgttgcaaatggagtgtgtcatatgctagcacctttatccactacctatgttggattctgtgtctatgcgggattctttggatttgccttcgggtggctcagctccgtattgtttgaaacattgatggaccttgttggaccccagaggttctccagcgctgtgggattggtgaccattgtggaatgctgtcctgtcctcctggggccaccacttttaggtcggctcaatgacatgtatggagactacaaatacacatactgggcatgtggcgtcgtcctaattatttcaggtatctatctcttcattggcatgggcatcaattatcgacttttggcaaaagaacagaaagcaaacgagcagaaaaaggaaagtaaagaggaagagaccagtatagatgttgctgggaagccaaatgaagttaccaaaacagcagaatctccggaccagaaagacacagaaggagggcccaaggaggaggaaagtccagtctgaatccatggggctgaagggtaaattgagcagttcatgacccaggatatctgaaaatattctactggcctgtaatctaccagtggtgctcaatgcaaatagtagacatttgtgtggaaatcataccagttgttcattgatgggatttttgtttgactccttaccaatagcctgaatttgaggagggaatgattggtagcaaaggatgggggaaagaagtaggttctgttttgttttgttttaatcttagcttttaatagtgtcataaagattataatatgtgccttaagttttagtctttagaactctagagagccttaacttcttaaaccatttttgctgaattcatctatttcgagtgttgtgttaaaaggaaaaataacaactaacttgtttgaggcaaatctaaaatttaaaattaatcttgcttcattgttacatgtaatatatttcagacattttcactggaagatttatgaacagaaatattggttgaaagttagagattttacaaaatgctgacaaaaatattttcctagcatcagtagatttctggcatatgtttctgctagctatatatttaggaaattcaaagcataaaactttggcaacatcttggctgttctagacacagtgtacttgtcaacccctctcaggtaccttttcttgggatgcttattagaagccaagtaaagtgcttaaggtttgttttcattaaattagctatttctgctcccctgttcaaagatgcattttgagtgtttatagatcactgccctttttgaaatcacctggtattatttttcttactggaaaagttagtattaaaatctacagaactacatatttgtgcctccttggtaaatacaacacatctaattaaatgtagacagatatttcaaacatcagctgaattcacttaagtttttccaaaacctcagttaaactgtgaagctattggaatttttttttcctggaatttttcccctttgattcacagtggtcccatttatatctgcttctagcttagtgctatgtgtgagatatgtgtgtgtttggtgttttt

SEQ ID NO: 14 - protein, 500 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKTAESPDQKDTEGGPKEEESPV

SEQ ID NO: 15 - DNA, 22 bp, Artificial Sequence
  Primer
ctccggaccagaaagacacagc

SEQ ID NO: 16 - DNA, 20 bp, Artificial Sequence
  Primer
gaagttaaggctctctagag

(19) United States
(12) Patent Application Publication
(10) Pub. No.: US 2003/0157484 A1
(43) Pub. Date: Aug. 21, 2003

(54) Chemical compounds

(76) Inventors: Carl John Cresswell, Macclesfield (GB); Adam Jeston Dudley, Wilmington, DE (US)

Correspondence Address: FISH & RICHARDSON PC, 225 FRANKLIN ST, BOSTON, MA, 02110, US

(21) Appl. No.: 09/925,641

(22) Filed: Aug. 10, 2001

Related U.S. Application Data
(60) Provisional application No. 60/233,624, filed on Sep. 18, 2000.

(30) Foreign Application Priority Data
Aug. 22, 2000 (GB) 0020544.3

(51) Int. Cl. C12Q 1/68; A61K 31/366

(52) U.S. Cl. 435/6; 514/460

(57) ABSTRACT

This invention relates to polymorphisms in the human MCT-1 gene. The invention also relates to methods and materials for analysing allelic variation in the MCT-1 gene, and to the use of MCT-1 polymorphism in treatment of diseases with MCT-1 transportable drugs.

This invention relates to polymorphisms in the human MCT-1 gene and corresponding novel allelic polypeptides encoded thereby. The invention also relates to methods and materials for analysing allelic variation in the MCT-1 gene, and to the use of MCT-1 polymorphism in treatment of diseases with MCT-1 transportable drugs.

Monocarboxylic acids play a major role in the metabolism of all cells, with lactic acid, the end product of glycolysis, being especially important. Some tissues, such as white skeletal muscle, red blood cells and many tumour cells, rely on this pathway to produce most of their ATP under normal physiological conditions, while all tissues become dependent on this pathway during hypoxia or ischaemia. Glycolysis produces two molecules of lactic acid for every glucose molecule consumed, and these must be transported out of the cell if high rates of glycolysis are to be maintained. If efflux of lactic acid from the cell does not keep pace with production, intracellular concentrations increase and cause the pH of the cytosol to decrease. This leads to inhibition of phosphofructokinase and hence glycolysis. Other tissues, such as brain, heart and red skeletal muscle, readily oxidize lactic acid, which may become a major respiratory fuel under some conditions. In these tissues lactic acid must be rapidly transported into the cell. The same is true for tissues such as the liver, which, through the operation of the Cori cycle, utilise lactate as their dominant gluconeogenic substrate (Denton, R. M. and Halestrap, A. P. (1979) Essays Biochem. 15, 37-47: Juel, C. (1997) Physiol. Rev. 77, 321-358.) Although it is lactic acid that is both produced and utilized by metabolism, the pK of lactic acid is 3.86, which ensures that it dissociates almost entirely to the lactate anion at physiological pH. This charged species cannot cross the plasma membrane by free diffusion, but requires a specific transport mechanism, provided by proton-linked monocarboxylate transporters (MCTs). These transporters catalyse the facilitated diffusion of lactate with a proton. There is no energy input other than that provided by the concentration gradients of lactate and protons, although the latter, in the form of a pH gradient, can drive the accumulation or exclusion of the lactate anion (Poole, R. C. and Halestrap, A. P. (1993) Am. J. Physiol. 264, C761-C782.: Juel, C. (1997) Physiol. Rev. 77, 321-358). A polymorphism Glu490Asp in MCT1 has been described by Merezhinskaya (2000), Muscle and Nerve, 23, 90-97.

MCT-1 is thought to be involved in the transport of drugs involved in lipid lowering e.g. statins. Statins have been referred to as a first-line therapy for patients with atherosclerotic vascular diseases.

All positions herein of polymorphisms in the MCT-1 polynucleotide relate to the position in SEQ ID NO 13 unless stated otherwise or apparent from the context.

All positions herein of polymorphisms in the MCT-1 polypeptide relate to the position in SEQ ID NO 14 unless stated otherwise or apparent from the context.

One approach is to use knowledge of polymorphisms to help identify patients most suited to therapy with particular pharmaceutical agents (this is often termed “pharmacogenetics”) . Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature) , position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The present invention is based on the discovery of polymorphisms in MCT-1. In particular, we have found two single nucleotide polymorphisms (SNPs) in the MCT-1 gene.

According to one aspect of the present invention there is provided a method for the diagnosis of a polymorphism in MCT-1 in a human, which method comprises determining the sequence of the nucleic acid of the human at at least one polymorphic position selected from one or more of the following positions:

positions 1450 and 2461 in the polynucleotide sequence of the MCT-1 gene as defined by the position in SEQ ID NO: 13.

The term human includes both a human having or suspected of having a MCT-1 mediated disease and an asymptomatic human who may be tested for predisposition or susceptibility to such disease. At each position the human may be homozygous for an allele or the human may be a heterozygote.

In one embodiment of the invention preferably the method for diagnosis described herein is one in which the single nucleotide polymorphism at position 1450 is presence of A and/or G.

In another embodiment of the invention preferably the method for diagnosis described herein is one in which the single nucleotide polymorphism at position 2461 is the presence of A and/or G.

The method for diagnosis is preferably one in which the sequence is determined by a method selected from amplification refractory mutation system and restriction fragment length polymorphism.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| FRET | Fluorescence resonance energy transfer |
| HMG-CoA | 3-hydroxy-3-methylglutaryl-coenzyme A |
| LCR | Ligase chain reaction |
| MASDA | Multiple allele specific diagnostic assay |
| MCT-1 | monocarboxylate transporter, human |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

Table 1—Mutation Detection Techniques

General: DNA sequencing, Sequencing by hybridisation

Scanning: PTT*, SSCP, DGGE, TGGE, Cleavase, Heteroduplex analysis, CMC, Enzymatic mismatch cleavage

*Note: not useful for detection of promoter polymorphisms.

Hybridisation Based

Solid phase hybridisation: Dot blots, MASDA, Reverse dot blots, Oligonucleotide arrays (DNA Chips)

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)

Extension Based: ARMS™, ALEX™—European Patent No. EP 332435 B1 (Zeneca Limited), COPS—Gibbs et al (1989), Nucleic Acids Research , 17, 2 347.

Incorporation Based: Mini-sequencing, APEX

Restriction Enzyme Based: RFLP, Restriction site generating PCR

Ligation Based: OLA

Other: Invader assay

Table 2—Signal Generation or Detection Systems

Fluorescence: FRET, Fluorescence quenching, Fluorescence polarisation—United Kingdom Patent No. 2228998 (Zeneca Limited)

Other: Chemiluminescence, Electrochemiluminescence, Raman, Radioactivity, Colorimetric, Hybridisation protection assay, Mass spectrometry

Table 3—Further Amplification Methods

SSR, NASBA, LCR, SDA, b-DNA

Preferred mutation detection techniques include ARMSTM, ALEX™, COPS, Taqman, Molecular Beacons, RFLP, and restriction site based PCR and FRET techniques.

Particularly preferred methods include ARMSTM and RFLP based methods. ARMSTM is an especially preferred method.

In a further aspect, the diagnostic methods of the invention are used to assess the pharmacogenetics of a drug transportable by MCT-1.

Assays, for example reporter-based assays, may be devised to detect whether one or more of the above polymorphisms affect transcription levels and/or message stability.

Individuals who carry particular allelic variants of the MCT-1 gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The diagnostic methods of the invention may be useful both to predict the clinical response to such agents and to determine therapeutic dose.

In a further aspect, the diagnostic methods of the invention, are used to assess the predisposition and/or susceptibility of an individual to diseases mediated by MCT-1. This may be particularly relevant in the development of hyperlipoproteinemia and cardiovascular disease and the present invention may be used to recognise individuals who are particularly at risk from developing these conditions.

In a further aspect, the diagnostic methods of the invention are used in the development of new drug therapies which selectively target one or more allelic variants of the MCT-1 gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes.

According to another aspect of the present invention there is provided a human MCT-1 gene or its complementary strand comprising a variant allelic polymorphism at one or more of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 bases of the human MCT-1 gene and comprising an allelic variant selected from any one of the following:

| | Position in SEQ |
|---|---|
| variant | ID NO 13 |
| G | 1450 |
| G | 2461 |

According to another aspect of the present invention there is provided a human MCT-1 gene or its complementary strand comprising an allelic variant, preferably corresponding with one or more the positions defined herein or a fragment thereof of at least 20 bases comprising at least one allelic variant.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting a MCT-1 gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a MCT-1 gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a MCT-1 gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the single nucleotide polymorphisms of this invention may be used as genetic markers in linkage studies. This particularly applies to the polymorphisms of relatively high frequency. The MCT-1 gene is on chromosome chromosome 1p13.2-p12. Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2n haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel single nucleotide polymorphisms in the vWF gene promoter. Blood (1999) 93:4277-83, Keightley A M, Lam Y M, Brady J N, Cameron C L, Lillicrap D).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

According to another aspect of the present invention there is provided a method of treating a human in need of treatment with a drug transportable by MCT-1 in which the method comprises:

i) diagnosis of a polymorphism in MCT-1 in the human, which diagnosis comprises determining the sequence of the human at one or more of the following positions: positions 1450, 1482 and 2461 in the sequence of the MCT-1 polynucleotide as defined by the position in SEQ ID NO: 13; and

at position 490 of human MCT-1 polypeptide as defined by the position in SEQ ID NO 14; and

ii) administering an effective amount of the drug.

Preferably determination of the status of the human is clinically useful. Examples of clinical usefulness include deciding which statin drug or drugs to administer and/or in deciding on the effective amount of the statin drug or drugs. Statins already approved for use in humans include atorvastatin, cerivastatin, fluvastatin, pravastatin and simvastatin. The reader is referred to the following references for further information: Drugs and Therapy Perspectives (May 12, 1997), 9: 1-6; Chong (1997) Pharmacotherapy 17: 1157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368. A preferred statin drug is compound 3a (S-4522) in Watanabe (1997) Bioorganic and Medicinal Chemistry 5: 437-444, presently known as rosuvastatin (Olsson, 2001, The American Journal of Cardiology, 87, 33-36). The term “drug transportable by MCT-1” means that transport by MCT-1 in humans is an important part of a drug exerting its pharmceutical effect in man. For example, some statins have to be transported to the liver by MCT-1 to exert their lipid lowering effects.

According to another aspect of the present invention there is provided use of a drug transportable by MCT-1 in preparation of a medicament for treating a disease in a human diagnosed as having a single nucleotide polymorphism therein, preferably at one or more of the positions defined herein. Preferably the disease is cardiovascular.

According to another aspect of the present invention there is provided a pharmaceutical pack comprising a MCT-1 transportable drug and instructions for administration of the drug to humans diagnostically tested for a single nucleotide polymorphism therein, preferably at one or more of the positions defined herein.

The invention will now be illustrated but not limited by reference to the following Examples. All temperatures are in degrees Celsius.

In the Examples below, unless otherwise stated, the following methodology and materials have been applied.

AMPLITAQ™,available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989).

Electropherograms were obtained in a standard manner: data was collected by ABI377 data collection software and the wave form generated by ABI Prism sequencing analysis (2.1.2).

### EXAMPLE 1

### Identification of Polymorphisms

1. Methods

DNA Preparation

DNA was prepared from frozen blood samples collected in EDTA following protocol I (Molecular Cloning: A Laboratory Manual, p392, Sambrook, Fritsch and Maniatis, 2^{nd} Edition, Cold Spring Harbor Press, 1989) with the following modifications. The thawed blood was diluted in an equal volume of standard saline citrate instead of phosphate buffered saline to remove lysed red blood cells. Samples were extracted with phenol, then phenol/chloroform and then chloroform rather than with three phenol extractions. The DNA was dissolved in deionised water.

Template Preparation

Templates were prepared by PCR using the oligonucleotide primers and annealing temperatures set out below. The extension temperature was 72° and denaturation temperature 94°. Generally 50 ng of genomic DNA was used in each reaction and subjected to 35 cycles of PCR. Where described below, the primary fragment was diluted 1/100 and two microlitres were used as template for amplification of secondary fragments. PCR was performed in two stages (primary fragment then secondary fragment) to ensure specific amplification of the desired target sequence.

Positions relate to SEQ ID NO 13

| Fragment | Forward Oligo | Reverse Oligo | Annealing Temp | Time |
|---|---|---|---|---|
| 13-578 | 13-32 | 559-578 | 58° C. | 60 s |
| 429-967 | 429-448 | 948-967 | 58° C. | 60 s |
| 778-1428 | 778-797 | 1409-1428 | 58° C. | 60 s |
| 1243-1837 | 1243-1262 | 1818-1837 | 58° C. | 60 s |
| 1667-2149 | 1667-1786 | 2130-2149 | 58° C. | 60 s |
| 2051-2556 | 2051-2070 | 2537-2556 | 58° C. | 60 s |

For dye-primer sequencing these primers were modified to include M13 forward and reverse primer sequences (ABI protocol P/N 402114, Applied Biosystems) at the 5′ end of the forward and reverse oligonucleotides respectively.

MCT1 oligos for cDNA Amplification

| | |
|---|---|
| Product 1F (13-32) | SEQ ID NO 1 |
| Product 1R (559-578) | SEQ ID NO 2 |
| Product 2F (429-448) | SEQ ID NO 3 |
| Product 2R (948-967) | SEQ ID NO 4 |
| Product 3F (778-797) | SEQ ID NO 5 |
| Product 3R (1428-1409) | SEQ ID NO 6 |
| Product 4F (1243-1262) | SEQ ID NO 7 |
| Product 4R (1837-1818) | SEQ ID NO 8 |
| Product 5F (1667-1786) | SEQ ID NO 9 |
| Product 5R (2149-2130) | SEQ ID NO 10 |
| Product 6F (2051-2070) | SEQ ID NO 11 |
| Product 6R (2556-2537) | SEQ ID NO 12 |

F=forward, R=reverse

Dye Primer Sequencing

Dye-primer sequencing using M13 forward and reverse primers was as described in the ABI protocol P/N 402114 for the ABI Prism™ dye primer cycle sequencing core kit with “AmpliTaq FS” DNA polymerase, modified in that the annealing temperature was 45 ° C. and DMSO was added to the cycle sequencing mix to a final concentration of 5%.

The extension reactions for each base were pooled, ethanol/sodium acetate precipitated, washed and resuspended in formamide loading buffer.

4.25% Acrylamide gels were run on an automated sequencer (ABI 377, Applied Biosystems).

Results

Polymorphisms

| Position | polymorphism | frequency |
|---|---|---|
| 1450 | A→G | 98% G |
| 1482 | A→T (Glu490Asp) | 46.7% A, 53.3% T |
| 2461 | A→G | 97.8% G, 2.2% A |

The allele frequencies were based on analysis of 23 individuals. The polymorphism Glu490Asp has been described by Merezhinskaya (2000), Muscle and Nerve, 23, 90-97.

### EXAMPLE 2

### Diagnostic Assay by Engineered RFLP for Polymorphism at Position 1482 of MCT-1

Methods (DNA and Template Preparation as in Example 1)

| | | |
|---|---|---|
| Diagnostic Primer | 1460-1481 | SEQ ID NO 15 |
| Constant Primer | 1818-1837 | SEQ ID NO 16 |

The diagnostic primer contains a single mismatch from the wild type sequence at the 3′ residue (A→C).

PCR amplification using these primers will generate a product of 377 bp. The use of the diagnostic primer on a template creates a PvuII recognition sequence at the site of the polymorphism. PvuII (New England Biolabs) can therefore distinguish the two polymorphic variants.

| | | |
|---|---|---|
| . . . ACA.CAG.CAG . . . | wild type | uncut |
| . . . ACA.CAG.CTG . . . | variant | cut |

1. A method for the diagnosis of a polymorphism in MCT-1 in a human, which method comprises determining the sequence of the nucleic acid of the human at at least one polymorphic position selected from one or more of the following positions:
positions 1450 and 2461 in the polynucleotide sequence of the MCT-1 gene as defined by the position in SEQ ID NO: 13.

2. A method according to claim one in which the polymorphisms are further defined as follows:
at position 1450 is presence of A and/orG; and
at position 2461 is the presence of A and/or G.

3. A method for diagnosis according to claim 1 or 2 in which the sequence is determined by a method selected from amplification refractory mutation system and restriction fragment length polymorphism.

4. A polynucleotide comprising at least 20 bases of the human MCT-1 gene and comprising an allelic variant selected from any one of the following:

| | Position in SEQ |
|---|---|
| variant | ID NO 13 |
| G | 1450 |
| G | 2461 |

5. An allele specific primer capable of detecting a MCT-1 gene polymorphism at one or more of the positions as defined in claim 1.

6. An allele-specific oligonucleotide probe capable of detecting a MCT-1 gene polymorphism at one or more of the positions defined in claim 1.

7. A diagnostic kit comprising an allele specific oligonucleotide probe as defined in claim 6 and/or an allele-specific primer as defined in claim 5.

8. Use of a polymorphism as defined in claim 1 as a genetic marker in linkage studies.

9. A computer readable medium comprising at least one variant sequence as defined in claim 4 stored on the medium.

10. A method of treating a human in need of treatment with a drug transportable by MCT-1 in which the method comprises:
i) diagnosis of a polymorphism in MCT-1 in the human, which diagnosis comprises determining the sequence of the human at one or more of the following positions: positions 1450, 1482 and 2461 in the sequence of the MCT-1 polynucleotide as defined by the position in SEQ ID NO: 13; and
at position 490 of human MCT-1 polypeptide as defined by the position in SEQ ID NO 14; and
ii) administering an effective amount of the drug.

11. A method according to claim 10 in which the drug is a statin.

12. A method according to claim 10 in which the drug is rosuvastatin.
